(19) United States
(12) Patent Application Publication
(10) Pub. No.: US 2026/0295142 A1
(43) Pub. Date: Oct. 1, 2026

(54) Drainage Apparatus, Disposable Collection Bag, Tubing Set, And Methods Of Use

(71) Applicant: Bard Peripheral Vascular, Inc., Franklin Lakes, NJ (US)

(72) Inventors: Michael Plishka, Lake Villa, IL (US); Erin Schaus, Elk Grove Village, IL (US); Akiva Kirschner, Chicago, IL (US); Christine Kearney, Palatine, IL (US)

(21) Appl. No.: 19/478,703

(22) PCT Filed: Apr. 26, 2024
(86) PCT No.: PCT/US2024/026591

§ 371 Date: Oct. 24, 2025

Related U.S. Application Data
(60) Provisional application No. 63/462,084, filed on Apr. 26, 2023.

(51) Int. Cl. A61M 1/00

(52) U.S. Cl. CPC A61M 1/82; A61M 2205/8206; A61M 2210/101; A61M 2210/1017

(57) ABSTRACT

A device may include an elongated catheter passage portion configured to have a distal end in a pleural or peritoneal cavity with a proximal end outside of the pleural or peritoneal cavity. A device may include a port apparatus at a proximal end of the elongated catheter, wherein the port has a connector interface for connection of a drainage device thereto and is configured to provide fluid communication with the catheter via a valve.

### CROSS REFERENCE TO RELATED APPLICATIONS

The present application claims the benefit of priority to U.S. Provisional Application No. 63/462084 entitled “HANDHELD DRAINAGE APPARATUS, DISPOSABLE COLLECTION BAG, TUBING SET, AND METHODS OF USE THEREOF,” filed on Apr. 26, 2023, the entirety of which is hereby incorporated by reference in the entirety for all purposes.

### TECHNICAL FIELD

This disclosure is related to a system for drainage of fluids from a patient. More specifically, a fluid pumping device, drainage bag and/or tubing set for the drainage of bodily fluids.

### BACKGROUND

The current options for pleural or peritoneal drainage typically involve gravity-based drainage and/or rely on an evacuated bottle or container or manual pumping by a user. There are a number of drainage systems for managing recurrent pleural effusions and ascites at home. One such system includes a drainage catheter and drainage bottles that collect fluid. The drainage catheter is inserted, typically as a simple outpatient procedure, in the chest for draining pleural effusions or in the abdomen for ascites. An end of the drainage catheter stays outside of the body, hidden under a bandage when not in use. To drain, the end of the drainage catheter is connected to a drainage line on the bottle that is pre-evacuated to provide a negative pressure, and the negative pressure inside the bottle is used to automatically draw out the bodily fluid.

### SUMMARY

The following presents a simplified summary of one or more aspects of the technology disclosed herein in order to provide a basic understanding of such aspects. This summary is not an extensive overview of all contemplated aspects and is intended to neither identify key or critical elements of all aspects nor delineate the scope of any or all aspects. Its sole purpose is to present some concepts of one or more aspects in a simplified form as a prelude to the more detailed description that is presented later.

Disclosed herein is a system for managing pleural or peritoneal fluid of a patient, the system including: a connector configured to connect to a pleural or peritoneal catheter; an elongated tubular portion extending from the connector with a lumen therein extending from a distal end of the tubular portion at the connector to a proximal end of the tubular portion; a fluid container configured to be in fluid communication with the elongated tubular portion and to contain collected fluid therein; and a pumping portion for pumping fluid from the pleural or peritoneal catheter to the fluid container. In some examples, the connector, elongated tubular portion, pumping portion, and fluid container all may be permanently connected and configured to be disposed of after use.

Additional aspects described herein include a method of managing or draining pleural or peritoneal fluid of a patient. The method includes connecting a connector to pleural or peritoneal catheter, wherein the connector is in fluid communication with an elongated tubular portion extending from the connector with a lumen therein extending from a distal end of the tubular portion at the connector to a proximal end of the tubular portion; pumping fluid from the tubular portion to a fluid container configured to be in fluid communication with the elongated tubular portion via a pumping portion; and controlling the pumping portion to pump fluid from the peritoneal or pleural catheter. The method may include removing the connector from the peritoneal or pleural catheter when a drainage operation is complete and disposing of the connector, the tubular portion, the pumping portion and the fluid container when the drainage operation is complete.

Additional aspects described herein include a method of making a system for managing pleural or peritoneal fluid of a patient, the system including: a connector configured to connect to a pleural or peritoneal catheter; an elongated tubular portion extending from the connector with a lumen therein extending from a distal end of the tubular portion at the connector to a proximal end of the tubular portion; a fluid container configured to be in fluid communication with the elongated tubular portion and to contain collected fluid therein; and a pumping portion for pumping fluid from the pleural or peritoneal catheter to the fluid container. In some examples, the connector, elongated tubular portion, pumping portion, and fluid container all may be permanently connected and configured to be disposed of after use.

To the accomplishment of the foregoing and related ends, the one or more aspects of the disclosure comprise the features hereinafter fully described and particularly pointed out in the claims. The following description and the annexed drawings set forth in detail include certain illustrative features of the one or more aspects. These features are indicative, however, of but a few of the various ways in which the principles of various aspects can be employed, and this description is intended to include all such aspects and their equivalents.

### BRIEF DESCRIPTION OF THE DRAWINGS

Features, characteristic of aspects of the technology described herein are set forth as follows, in the appended claims, and in the drawings. In the descriptions that follow, like parts are marked throughout the specification and drawings with the same numerals, respectively. The drawing figures are not necessarily drawn to scale and certain figures can be shown in exaggerated or generalized form in the interest of clarity and conciseness. The disclosure itself, however, as well as a preferred mode of use, further objects and advances thereof, will be best understood by reference to the following detailed description of illustrative aspects when read in conjunction with the accompanying drawings.

FIG. 1 shows one example of a drainage system and device according to aspects of the disclosure.

FIG. 2 shows an alternative example of a drainage system and device according to aspects of the disclosure.

FIG. 3A shows an example of a first pumping mechanism usable with aspects of the disclosure.

FIG. 3B shows an example of a second pumping mechanism usable with aspects of the disclosure.

FIG. 4A shows an example of a third pumping mechanisms usable with aspects of the disclosure.

FIG. 4B shows an example of a fourth pumping mechanism usable with aspects of the disclosure.

FIG. 5 shows an example representative diagram of various components of example controllers usable with aspects of the disclosure.

FIG. 6 shows an example of a computer system in accordance with aspects of the disclosure;

FIG. 7 shows an example of various system components in accordance with aspects of the present disclosure.

FIG. 8 shows an example of various system features in accordance with aspects of the present disclosure.

### DETAILED DESCRIPTION

The detailed description set forth below in connection with the appended drawings is intended as a description of various configurations and is not intended to represent the only configurations in which the concepts described herein can be practiced. The detailed description includes specific details for the purpose of providing a thorough understanding of various concepts. However, it will be apparent to those skilled in the art that these concepts can be practiced without these specific details. In some instances, well known structures and components are shown in block diagram form in order to avoid obscuring such concepts.

### I. Terminology

Reference throughout this specification to one aspect, an aspect, one example or an example means that a particular feature, structure or characteristic described in connection with the embodiment or example may be a feature included in at least example of the present disclosure. Thus, appearances of the phrases in one aspect, in an aspect, one example or an example in various places throughout this specification are not necessarily all referring to the same example. Furthermore, the particular features, structures or characteristics may be combined in any suitable combinations and/or subcombinations in one or more embodiments or examples.

Throughout the disclosure, the terms substantially or approximately may be used as a modifier for a geometric relationship between elements or for the shape of an element or component. While the terms substantially or approximately are not limited to a specific variation and may cover any variation that is understood by one of ordinary skill in the art to be an acceptable level of variation, some examples are provided as follows. In one example, the term substantially or approximately may include a variation of less than 10% of the dimension of the object or component. In another example, the term substantially or approximately may include a variation of less than 5% of the object or component. If the term substantially or approximately is used to define the angular relationship of one element to another element, one non-limiting example of the term substantially or approximately may include a variation of 5 degrees or less. These examples are not intended to be limiting and may be increased or decreased based on the understanding of acceptable limits to one of skill in the relevant art.

For purposes of the disclosure, directional terms are expressed generally with relation to a standard frame of reference when the aspects or articles described herein are in an in-use orientation. In some examples, the directional terms are expressed generally with relation to a left-hand coordinate system.

Terms such as a, an, and the, are not intended to refer to only a singular entity, but also include the general class of which a specific example may be used for illustration. The terms a, an, and the, may be used interchangeably with the term at least one. The phrases at least one of and comprises at least one of followed by a list refers to any one of the items in the list and any combination of two or more items in the list. All numerical ranges are inclusive of their endpoints and non-integer values between the endpoints unless otherwise stated.

The terms first, second, third, and fourth, among other numeric values, may be used in this disclosure. It will be understood that, unless otherwise noted, those terms are used in their relative sense only. In particular, certain components may be present in interchangeable and/or identical multiples (e.g., pairs). For these components, the designation of first, second, third, and/or fourth may be applied to the components merely as a matter of convenience in the description.

The terms disposed on, disposed along, disposed with, or disposed toward and variations thereof may define one element can be integral with another element, or that one element can be a separate structure bonded to or placed with or placed near another element.

The term at, for example when referring to something being located at a specific location, is intended to include any one or more of: proximate, on, near, adjacent to or within the specific location. As used herein, the terms proximal is defined with respect to an object, element, or user. For example, the term proximal may refer to the part or portion closer to the user.

### II. Overview

This overview, and the detailed description that follows, has been presented for purposes of illustration and description. It is not intended to be exhaustive nor to limit the disclosure to the forms described. Numerous modifications are possible considering the teachings herein, including any combination of the different examples described herein. Some of those modifications have been discussed and others will be understood by those skilled in the art. The various aspects were chosen and described to best illustrate the principles of the present disclosure and various aspects as are suited to the particular use contemplated. The scope of the present disclosure is, of course, not limited to the examples or aspects set forth herein but can be employed in any number of applications and equivalent devices by those of ordinary skill in the art. Rather, it is hereby intended the scope be defined by the claims appended hereto.

The current options for pleural or peritoneal drainage catheters typically require that the catheter exit through the skin and terminate in a portion of the catheter that remains outside of the body. Some current options for pleural or peritoneal drainage typically involve gravity-based drainage and/or rely on an evacuated bottle or container or manual pumping by a user. Some additional examples require a pump that is connected in-line with tubing and/or that requires a technician to install a pump onto tubing. Further, for sanitary reasons, use of a pump may require specific fittings and/or techniques for sanitizing a fluid path within the pump.

As technology and understanding of pleural and peritoneal symptoms advances, more patients are able to enjoy prolonged life and independence despite dealing with pleural and peritoneal fluid build-up symptoms. In some cases, especially when using an evacuated bottle or container, patients report an increase in pain. One theory for this pain is that evacuated bottles or containers may apply excessive vacuum, especially at the start of a draining procedure. Vacuum levels, and flow rates, are largely out of control of the patient. Thus, aspects of the disclosure are related to systems and methods for improving a patient's ease and control of drainage-in some instances a drainage system described herein may be intuitive and simple enough to be used by a patient for pleural or peritoneal drainage while providing reduced risk of contamination by decreasing the number of steps required for drainage. In other aspects, a drainage system described herein may provide a technician with similar advantages.

Additional advantages include but are not limited to: less steps in a drainage procedure because a negative pressure system does not have to be maintained or protected; improved shipping/logistics due to more compact system that is not under pressure; the entire system can be packaged in a sanitized or high-level disinfected pouch or other packaging to allow ease of shipping and/or handling by a user while reducing a risk of contamination; a patient can perform drainage on their own because they would only be using a power button and/or a vacuum control interface and can control the vacuum, which may improve user comfort; the bag full of drainage fluid, pump, and/or tubing and connector can easily be handled/disposed of for safe and easy disposal at home; the described system may allow for advanced control of the drainage flow rate and monitoring/analytics; the system is compact and thus is easy to transport during travel; the system may be able to calculate volume drained via flow sensors and/or measure the properties of drained fluid if pH sensors, oxygen sensors, glucose sensors, optical transmitters/receivers are integrated into the system, which could provide information to the caregiver or clinician in real time regarding the state of the patient's disease; and a system as described herein can connect wirelessly to an external smart device for recording of drainage data and which can be accessed by patient, clinician or electronic medical records “EMR” system.

Additional details and examples and advantages are described in the non-limiting examples below.

### III. Detailed Examples

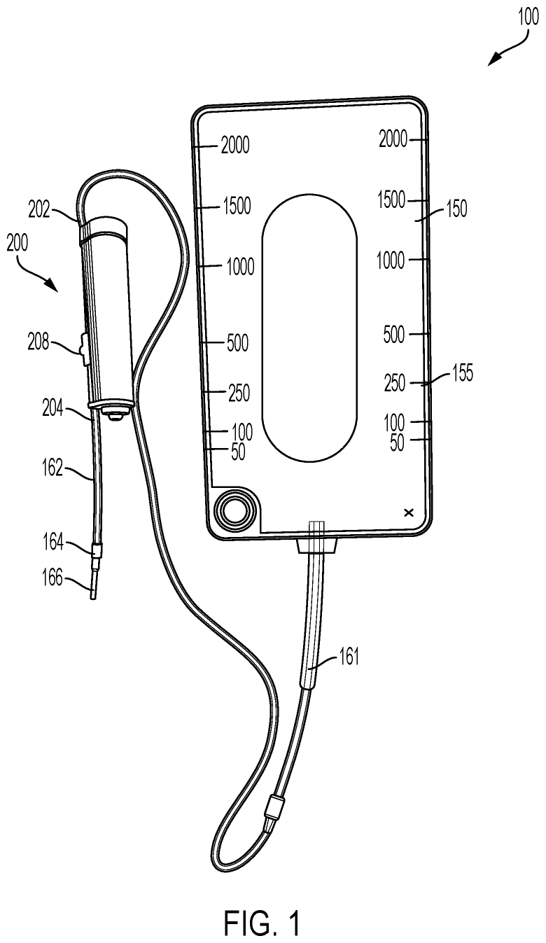

This disclosure describes a system and method to collect and/or assist with the collection of fluid from a body cavity, such as the pleural cavity or peritoneal cavity. The system may include one of or a combination of a disposable collection bag and tubing set, electrical pumping unit and/or an electro-mechanical pumping unit. FIG. 1 shows an example of a system 100 according to aspects of the disclosure. The system 100, may include a drainage container 150. In some examples the drainage container may be a bag, which may provide packaging and disposal advantages. However, it is noted that the drainage container 150 is not limited to a bag and may instead be a bottle or like container. In one example the drainage bag or container 150 may be filled or partially filled with a superabsorbent polymer to absorb drained liquid. Utilizing a superabsorbent polymer may further improve safety and ease of disposal by absorbing liquid and preventing potential leaks. The drainage container 150 may have graduated markings 155 thereon to patient or technician to recognize the volume of fluid contained therein.

The drainage container 150 may be connected to a pumping portion or apparatus 200 via a proximal section or first section of tubing 161. In some examples, the tubing 161 may be permanently connected (i.e., go discourage or prevent disconnection) at the outlet of drainage container 150 and/or at an outlet portion 202 of the pumping portion or apparatus 200. It is noted that throughout the disclosure, the terms pumping portion or pumping apparatus may be used interchangeably. A second section of tubing 162 may connect to a pumping apparatus inlet 204. In some examples, the second section of tubing 162 may be permanently connected to the pumping apparatus inlet (i.e., go discourage or prevent disconnection). The distal end of the tubing 162 may include a catheter connector 164 configured to connect with an indwelling catheter of a patient and provide fluid communication from the indwelling catheter of the patient and the tubing 162. In some examples, the catheter connector 164 may include a valve opening feature for opening an otherwise sealed valve of the indwelling catheter. In the non-limiting example shown in FIG. 1, the valve opening feature is shown as a dilator 166.

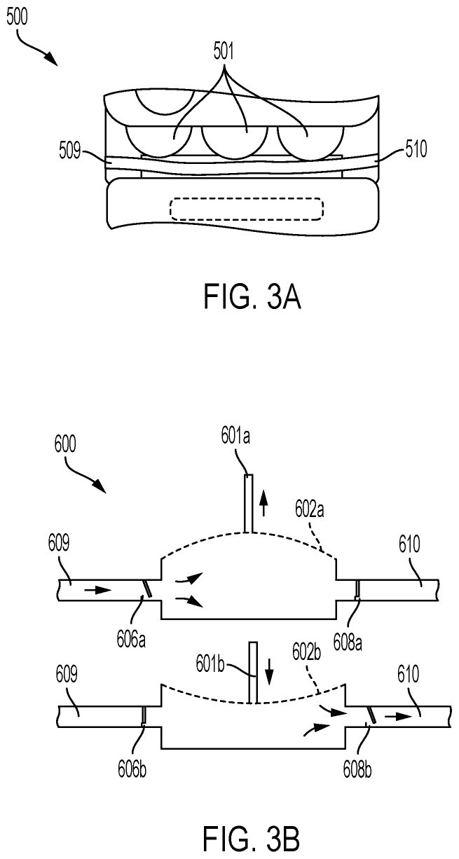

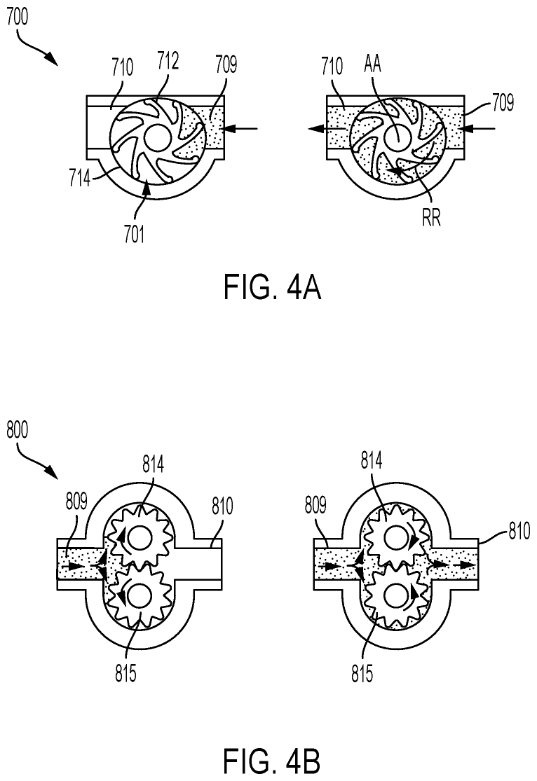

As noted above, the system 100 may include a pumping apparatus 200 configured to pump fluid from the catheter connector 164 into the drainage container 150. The pumping apparatus 200 may be self-contained and include a pumping apparatus therein that utilizes mechanical motion of an electric motor to pump fluid from the pumping apparatus inlet 204 to the outlet portion 202. Examples of the pumping apparatus contained within the pumping apparatus 200 as further described with respect to FIGS. 3A-4B below. The pumping apparatus 200 may further include a pump controller interface 208. In some examples, the pump controller interface 208 may include an on/off switch or like control. In some examples, the pump controller interface 208 may include a speed control slider or knob for controlling the speed (and thus the pumping force or pressure) of drainage. In the aforementioned example, the speed control slider or knob may additionally be used to turn on/off the pump (e.g., by turning or sliding the knob or slider to an end position). In another example, the pump controller interface 208 may include a separate on/off switch and any of the flow controllers disclosed herein. In another example, usable with or as an alternative to any of the aforementioned configurations, the pumping apparatus 200 may be wirelessly connectable to a phone or other smart device. An application loaded onto the phone or smart device may provide a graphical user interface (GUI) to allow a user to control the features described herein. Additional aspects related to the wireless connection is described in further detail below.

In one example, the pumping apparatus 200 may be compact sized so as to be easily held by a user and/or transported or stored. In one example, the pumping apparatus 200 may have length from 2-10 inches. In another example, the pumping apparatus 200 may have a length from 4-7 inches.

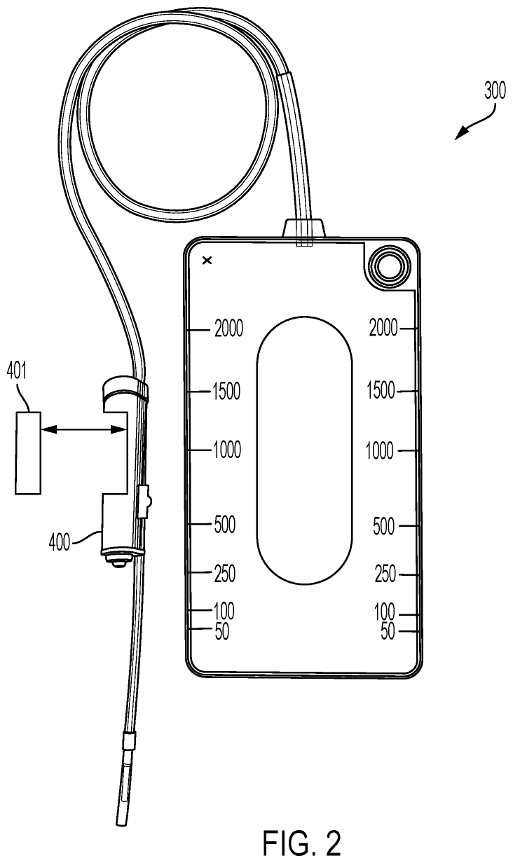

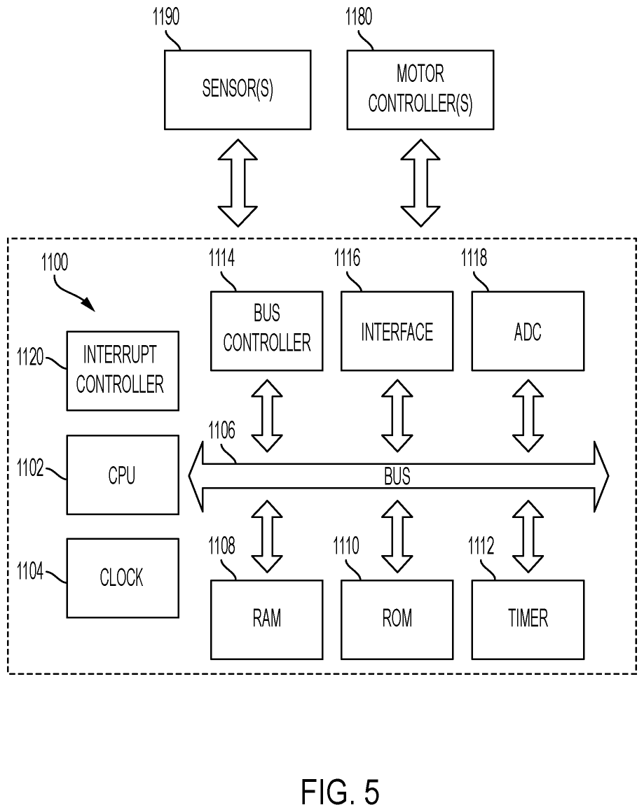

The pumping apparatus 200 may include a power source such as a battery enclosed within the pumping apparatus 200 for powering the motor and/or for providing power to control features and/or communication features which may be implemented into the pumping apparatus 200, which are described in further detail with reference to FIGS. 5-8 below. In some examples, the battery may be non-user replaceable (i.e., may be integrated the pumping apparatus 200 such that removal requires disassembly or destruction of the pumping apparatus 200). One example of a pumping apparatus 200 and drainage system 100 that includes a non-removable battery is shown in FIG. 1 as reference 200. In another example, the battery may be either removable/replaceable by a user or may be configured to be installed before use of the pumping apparatus 200. The aforementioned example may further improve shelf-life of the drainage system 300b and/or prevent premature depletion or corrosion of a battery prior to use. For example, as shown in FIG. 2B, a battery module 401 or battery door may be installed into a pumping apparatus 400b either prior to using the pumping apparatus 400b and/or as a replacement if a battery in the pumping apparatus 400b is depleted. Further, any of the aforementioned pumping apparatuses (e.g., 200, 200 and/or 400b) may further utilize a rechargeable battery and a charging port or other interface for recharging a battery of the pumping apparatus. In some examples, the battery may be a capacitor. In some examples, where pumping may occur with a lower volume, for example over a longer period of time, the battery may be charged via one or more energy harvesting devices that convert motion of the patient to mechanical motion used to generate electricity. For example, a rotating weight or freewheel may be implemented that rotates in response to body vibration and drives a generator.

Any one of the drainage systems described herein may utilize an indicator light 410 or other visual indicator that the pumping apparatus (e.g., pumping apparatus 200, 200, and/or 400b) to indicate a status of the pumping apparatus. In some examples, the visual indicator may provide an indication that the pump has power and/or that the pump is in a standby state and ready to drain fluid. A visual indicator (i.e., the same visual indicator and/or a secondary visual indicator) may indicate that the pump is currently pumping. Visual indicators may further be utilized to indicate a pump speed, battery status, and/or may indicate the output of one or more sensors for indicating a drainage state. It is noted that while a visual indicator is discussed above, a tactile (i.e., haptic) and/or audible indicator may be implemented either in combination with or as a substitute for any one or combination of the aforementioned visual indicator(s).

FIGS. 3A-4B show examples of pumping mechanisms that may be implemented into the pumping apparatus (e.g., pumping apparatus 200 and/or 400b) of the drainage system (e.g. drainage system 100 and/or 300b). As shown in FIG. 3, a roller or peristaltic system may be implemented to provide a pumping force or pressure difference to drain fluid as described above. In the example shown in FIG. 3A, the pumping device 500 may include a plurality of rollers 501 that are configured to pinch or otherwise compress a flexible tubing 502 along a length thereof. The one or more roller 501 may progressively provide a pinching or compressing force to the flexible tubing 502 thus forcing fluid within the flexible tubing 502 from an inlet (e.g., inlet 510) to an outlet (e.g. outlet 509) of the pumping device 500. One advantage of utilizing the pumping mechanism of FIG. 3A is that the flow-path of the fluid remains isolated from the interior of the pumping apparatus, which may prevent leaks and/or prevent contamination. Another advantage is that the mechanism of FIGS. 3A-3B can move thicker, more slurry like fluids without clogging.

FIG. 3B shows an example of a diaphragm pump that may be utilized in the pumping apparatus (e.g., pumping apparatus 200, 200 and/or 400b) of the drainage system (e.g. drainage system 100, 100 and/or 300b). The diaphragm pump 400 may utilize one or more flexible or semi-flexible diaphragms 602 driven in a first direction and a second direction (e.g., as indicated by the arrows in FIG. 3B) by a motor or the like. The diaphragm(s) may be within a flow path of the fluid between an inlet 609 and an outlet 610 of the pumping apparatus. The diaphragm pump 600 may further utilize an upstream check valve 606a, 606b and a downstream check valve 608a, 608b. As shown in the example of FIG. 3B, when a driven driving member is in a first position 601a (e.g., as indicated by the upward arrow), pulling the diaphragm into position 602a, fluid opens and is drawn through the open upstream check valve 606a. As the driven driving member is moved to a second position 602b in the direction indicated by the arrow, the increase in pressure causes the upstream check valve to move to a closed position 606b and causes the downstream check valve to move to the open position 608b a fluid is forced toward the outlet 610. The aforementioned process is repeated to pump fluid from the inlet 609 to the outlet 610. One advantage of utilizing the pumping mechanism of FIG. 3B is that the flow-path of the fluid can remain largely isolated from the interior of the pumping apparatus, which may prevent leaks and/or prevent contamination. Further a diaphragm pump can also have the advantage being able to move thicker, more slurry like fluids without clogging. Further, the pumping mechanism of FIG. 3B requires few moving parts that are simple in construction and operation, which may help reduce the cost of the system.

FIG. 4A shows another example of a pump apparatus that may be utilized in the pumping apparatus (e.g., pumping apparatus 200, 200 and/or 400b) of the drainage system (e.g. drainage system 100, 100 and/or 300b). The pump apparatus 700 of FIG. 4A has a fluid passage inlet 709 and a fluid passage outlet 710 and an impeller casing 714 with a rotatable impeller 701 configured to rotate therein. The impeller may include a plurality of paddles or blades 712. As indicated by the arrows, when the impeller is rotated via a motor for examples, fluid is pushed from the fluid passage inlet 709 to the fluid passage outlet 710 via the paddles or blades 712 of the rotatable impeller 701. In some examples, the impeller blades 712 may be formed of a flexible or elastomeric material and may be configured to sealingly contact a portion of an interior wall of the impeller casing 714 to improve efficiency of the pump apparatus 700. One advantage of utilizing the pumping mechanism of FIG. 4A is efficiency of pumping, further the pumping mechanism 700 of FIG. 4b requires few moving parts that are simple in construction and operation, which may help reduce the cost of the system.

Another example pump apparatus is shown in FIG. 4B. The pump 800 in FIG. 4B is an example of a rotary displacement pump that may be utilized in the pumping apparatus (e.g., pumping apparatus 200, 200 and/or 400b) of the drainage system (e.g. drainage system 100, 100 and/or 300b). The pump 800 may include a first gear or paddles 814 and a second gear or paddles 815 that are configured to rotatably intermesh to force fluid from a pump inlet 809 to a pump outlet 810 as indicated by the arrows shown in FIG. 4B. One advantage of utilizing the pumping mechanism of FIG. 4A is efficiency of pumping, further the pumping mechanism 700 of FIG. 4A requires few moving parts that are simple in construction and operation, which may help reduce the cost of the system. Further, the rotary displacement pump shown in FIG. 4B may provide a smooth flow with little pulsation and/or a low internal volume that may provide for a reliable measure of liquid passing through a pump and hence accurate flow control.

Typically, drainage procedures remove 1 to 2 liters of fluid in a single session, offering relief to the patient. This large volume of drainage must be done while seated or reclining and it carries the risk of pain. However, in some cases, extending the drainage over a longer period—using smaller volume pumps with lower flow rates—allows patients to maintain mobility and live their lives without being confined to a chair and table during the drainage process. This also prevents the greater accumulation of fluid in the body which can lead to discomfort. In some examples, the pumping apparatus (e.g., pumping apparatus 200, 200 and/or 400b) may have a low-flow rate to spread out drainage and potentially prevent pain that may be experienced drainage. In one example, the flow rate of the pump is from 3ml/min to 400 ml/min. In yet another example, the flow rate of the pump is from 200 ml/min to 350 ml/min. In another example, the flow rate of the pump may be from 10 ml/min to 100 ml/min.

The drainage system described above, for example the system 100 and/or 300b may come pre-packaged in a sealed pouch, optionally with any additional components or supplies that are helpful during a drainage procedure. In one aspect, the system 100 and/or 300b may be packaged in a sealed pouch and sterilized or high-level disinfected, thus ensuring the contents remain sterilized or high-level disinfected until a user opens the pouch. In some examples, the system 100 and/or 300b unit and/or collection bag and tubing could also be supplied with or configured to be placed or otherwise contained within a fanny-pack like attachment to the body for wearable drainage.

Aspects of the disclosure further include method for draining fluid from a patient using the device and systems described herein. The method may comprise the following steps:
a. Connect disposable access tip and tubing to catheter
b. To begin draining, press power button, slide slider, or turn knob on pumping mechanism or on phone application
c. To control drainage flow rate, adjust via buttons, slider, and/or knob on pumping device and/or phone application
d. To pause the drainage temporarily, press pump/pause button on pumping device or phone application. To resume drainage, press the pump/pause button again.
e. When finished draining, turn off the pump module. Disconnect access tip from catheter, seal disposable bag full of drainage fluid and dispose of system.

In one example, any one or combination of the features described herein could be labeled clearly with numbers and/or color coded and/or may include any combination of written and/or pictographic instructions thereon so that the user can comprehend the steps for installation and/or use of the device or features thereof.

In some examples, the aforementioned and/or a related procedure and/or method for using the system may be described in instructions provided with the device and any additional components included therewith (e.g., a catheter, drainage bag, the pumping unit, and/or any other components provide to assist with installation, use-of and/or maintenance of the devices or components). The instructions may be provided as a pamphlet or as written instructions on the packaging of the kit or system. The aforementioned instructions may also be provided as digital content (provided by accessing information on a network, such as a webpage on the Internet). In one example, any one of the component systems, the packaging of the system components and/or any components provided therewith may include a web address and/or a quick response “QR” code for accessing instructions and/or digital content related to methods of installation and/or use thereof. In addition, the instructions may also be provided by salesperson via marketing materials to name another non-limiting example.

The system 100 and/or 300b may further include one or more pressure sensor(s) to measure pressure in the system before, during, and/or after drainage. In one example, the output of the pressure sensor(s) may be used to control the operation of the pumping device (e.g., pumping apparatus 200 and/or 400b). For example, if the pressure exceeds a predetermined threshold or range, the pump speed may be decreased or stopped until the pressure again falls below the threshold or within a desired range.

The aforementioned system may additionally include flow rate sensors or volumetric sensors, pH sensors, oxygen sensors, glucose sensors, optical transmitters/receivers for monitoring the properties of the fluid being drained. In one example, the system may include biomarkers for determining white blood cell count, oxygenation, levels, glucose levels and/or other biomarkers. The output of the sensor(s) may be used to either help determine a condition of the patient and/or for data gathering for determining or otherwise monitoring health trends associated with such outputs. In some examples, any one or combination of the aforementioned sensors may be implemented into the pumping apparatus (e.g., pumping apparatus 200 and/or 400b). For example, one or more of the aforementioned sensors may be located at and/or within the flow path of the fluid that travels through the pumping apparatus.

In one example, the system (e.g., system 100 and/or 300b) may include any one or combination of the aforementioned sensors and monitoring systems integrated into the pumping apparatus (e.g., pumping apparatus 200 and/or 400b). In another example, the aforementioned pumping apparatus may wirelessly communicate with or otherwise communicate the status of the device and/or output data related to one or more sensor(s) to an external device (e.g., a smartphone or to the cloud) using a known system or via the aspects described below.

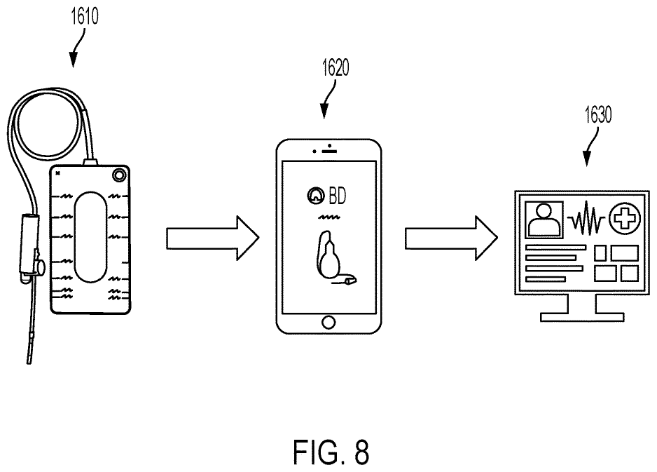

The aforementioned monitoring system and/or one or more monitoring sensor(s) could be integrated into or otherwise associated with the device or otherwise output and be integrated with an application or other software implemented method to measure any one or combination of the amount, flow-rate, and/or pressure of fluid that is collected. Any of the aforementioned aspects may be monitored with respect to time and/or may be monitored with respect to a patient's perceived pain or other patient observation(s). The aforementioned pumping unit's wireless connectivity may be used for transmission of acquired drainage data (such as flow rate, volume drained) from the pumping unit to an external smart device 1620 (FIG. 8). The smart device may have a custom-developed app to receive the data and store it in a drainage log. In addition, the app may provide feedback to the user on flow rate settings for future drainages and create custom drainage rate protocols for individual patients. The smartphone application or software may additionally send data to an electronic medical records “EMR” system 1630 that may be remotely viewed by a patient or authorized healthcare technician.

In one example, the aforementioned pumping apparatus may include readout screen for displaying any of the monitoring aspects described above. In one aspect the readout screen may also be an input device (i.e., a touchscreen).

In some examples, the output of the sensor(s) 1190 may be configured to provide information to and/or may otherwise be communicatively coupled with one or more microcontrollers. Further, in some examples, either in combination with the implementation f sensor(s) or as a separate configuration, one or more motor controller(s) 1180 may be implemented to control the flow rate of the pumping apparatus (e.g., pumping apparatus 200 and/or 400b). Various components of such a controller 1100 are shown in representative block diagram form in FIG. 5. In FIG. 5, the controller 1100 includes a CPU 1102, clock 1104, RAM 1108, ROM 1110, a timer 1112, a BUS controller 1114, an interface 1116, and an analog-to-digital converter (ADC) 1118 interconnected via a BUS 1106.

The CPU 1102 may be implemented as one or more single core or multi-core processors, and receive signals from an interrupt controller 1120 and a clock 1104. The clock 1104 may set the operating frequency of the entire microcontroller 1100 and may include one or more crystal oscillators having predetermined frequencies. Alternatively, the clock 1104 may receive an external clock signal. The interrupt controller 1120 may also send interrupt signals to the CPU, to suspend CPU operations. The interrupt controller 1120 may transmit an interrupt signal to the CPU when an event requires immediate CPU attention.

The RAM 1108 may include one or more Static Random Access Memory (SRAM), Dynamic Random Access Memory (DRAM), Synchronous Dynamic Random Access Memory (SDRAM), Double Data-Rate Random Access Memory (DDR SDRAM), or other suitable volatile memory. The Read-only Memory (ROM) 1110 may include one or more Programmable Read-only Memory (PROM), Erasable Programmable Read-only Memory (EPROM), Electronically Erasable Programmable Read-only memory (EEPROM), flash memory, or other types of non-volatile memory.

The timer 1112 may keep time and/or calculate the amount of time between events occurring within the controller 1100, count the number of events, and/or generate baud rate for communication transfer. The BUS controller 1114 may prioritize BUS usage within the controller 1100. The ADC 1118 may allow the controller 1100 to send out pulses to signal other devices.

The interface 1116 may comprise an input/output device that allows the controller 1100 to exchange information with other devices. In some implementations, the interface 1116 may include one or more of a USB port, parallel port, serial port, or other computer interfaces.

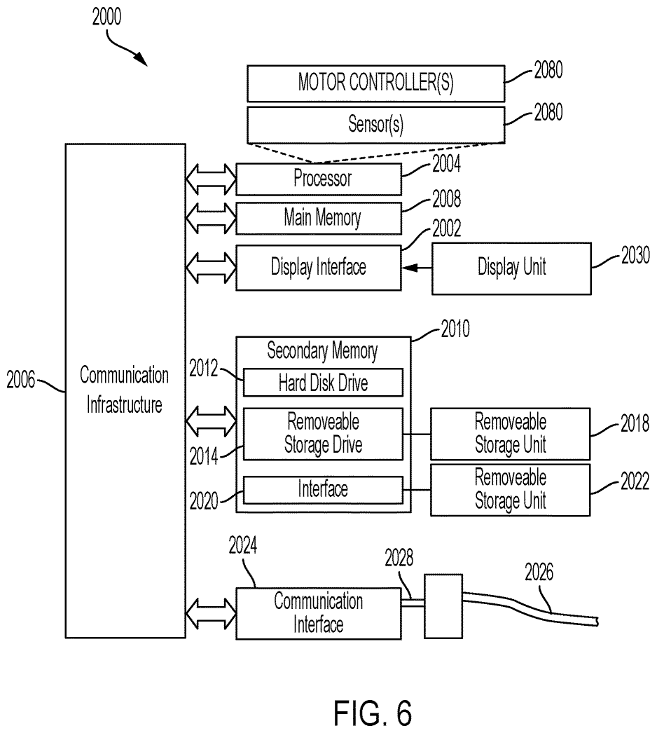

In addition, aspects of the present disclosures may be implemented using hardware, software, or a combination thereof and may be implemented in one or more computer systems or other processing systems. In an aspect of the present disclosures, features are directed toward one or more computer systems capable of carrying out the functionality described herein. An example of such a computer system 2000 is shown in FIG. 6.

The computer system 2000 may include one or more processors, such as processor 2004. The processor 2004 may be connected to a communication infrastructure 2006 (e.g., a communications bus, cross-over bar, or network). Various software aspects are described in terms of this example computer system. After reading this description, it will become apparent to a person skilled in the relevant art(s) how to implement aspects of the disclosures using other computer systems and/or architectures.

The computer system 2000 may include a display interface 2002 that forwards graphics, text, and other data from the communication infrastructure 2006 (or from a frame buffer not shown) for display on a display unit 2030, which may be analogous with the display interface 102. Computer system 2000 also includes a main memory 2008, preferably random-access memory (RAM), and may also include a secondary memory 2010. The secondary memory 2010 may include, for example, a hard disk drive 2012, and/or a removable storage drive 2014, representing a floppy disk drive, a magnetic tape drive, an optical disk drive, a universal serial bus (USB) flash drive, etc. The removable storage drive 2014 reads from and/or writes to a removable storage unit 2018 in a well-known manner. Removable storage unit 2018 represents a floppy disk, magnetic tape, optical disk, USB flash drive etc., which is read by and written to removable storage drive 2014. As will be appreciated, the removable storage unit 2018 includes a computer usable storage medium having stored therein computer software and/or data.

Alternative aspects of the present disclosure may include secondary memory 2010 and may include other similar devices for allowing computer programs or other instructions to be loaded into computer system 2000. Such devices may include, for example, a removable storage unit 2022 and an interface 2020. Examples of such may include a program cartridge and cartridge interface (such as that found in video game devices), a removable memory chip (such as an erasable programmable read only memory (EPROM), or programmable read only memory (PROM)) and associated socket, and other removable storage units 2022 and interfaces 2020, which allow software and data to be transferred from the removable storage unit 2022 to computer system 2000.

Computer system 2000 may also include a communications interface 2024. Communications interface 2024 allows software and data to be transferred between computer system 2000 and external devices. Examples of communications interface 2024 may include a modem, a network interface (such as an Ethernet card), a communications port, a Personal Computer Memory Card International Association (PCMCIA) slot and card, etc. Software and data transferred via communications interface 2024 are in the form of signals 2028, which may be electronic, electromagnetic, optical or other signals capable of being received by communications interface 2024. These signals 2028 are provided to communications interface 2024 via a communications path (e.g., channel) 2026. This path 2026 carries signals 2028 and may be implemented using wire or cable, fiber optics, a telephone line, a cellular link, an RF link and/or other communications channels. In this document, the terms “computer program medium” and “computer usable medium” are used to refer generally to media such as a removable storage drive 2018, a hard disk installed in hard disk drive 2012, and signals 2028. These computer program products provide software to the computer system 2000. Aspects of the present disclosures are directed to such computer program products.

Computer programs (also referred to as computer control logic) are stored in main memory 2008 and/or secondary memory 2010. Computer programs may also be received via communications interface 2024. Such computer programs, when executed, enable the computer system 2000 to perform the features in accordance with aspects of the present disclosures, as discussed herein. In particular, the computer programs, when executed, enable the processor 2004 to perform the features in accordance with aspects of the present disclosures. Accordingly, such computer programs represent controllers of the computer system 2000.

In an aspect of the present disclosures where the method is implemented using software, the software may be stored in a computer program product and loaded into computer system 2000 using removable storage drive 2014, hard drive 2012, or communications interface 2020. The control logic (software), when executed by the processor 2004, causes the processor 2004 to perform the functions described herein. In another aspect of the present disclosures, the system is implemented primarily in hardware using, for example, hardware components, such as application specific integrated circuits (ASICs). Implementation of the hardware state machine so as to perform the functions described herein will be apparent to persons skilled in the relevant art(s).

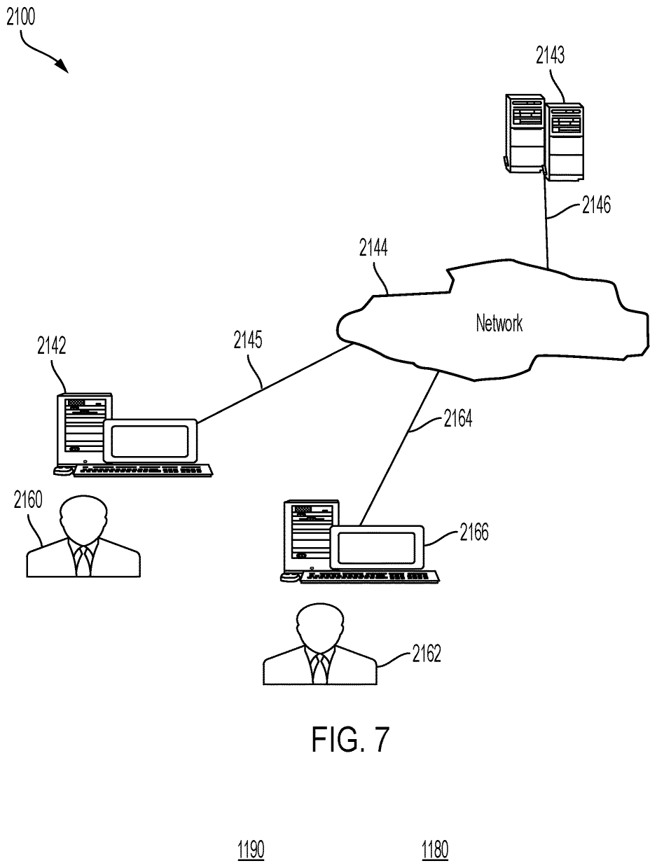

FIG. 7 is a block diagram of various example communication system components usable in accordance with an aspect of the present disclosure. The communication system 2100 includes one or more accessors 2160, 2162 (which may for example comprise any of the aforementioned systems and features) and one or more terminals 2142, 2166. In one aspect, data for use in accordance with aspects of the present disclosure is, for example, input and/or accessed by accessors 2160, 2162 via terminals 2142, 2166, such as personal computers (PCs), minicomputers, mainframe computers, microcomputers, telephonic devices, or wireless devices, such as personal digital assistants (“PDAs”) or a hand-held wireless devices coupled to a server 2143, such as a PC, minicomputer, mainframe computer, microcomputer, or other device having a processor and a repository for data and/or connection to a repository for data, via, for example, a network 2144, such as the Internet or an intranet, and couplings 2145, 2146, 2164. The couplings 2145, 2146, 2164 include, for example, wired, wireless, or fiberoptic links. In another example variation, the method and system in accordance with aspects of the present disclosure operate in a stand-alone environment, such as on a single terminal.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described herein that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

1. A system for managing pleural or peritoneal fluid of a patient, the system comprising: a connector configured to connect to a pleural or peritoneal catheter;
connector configured to connect to a pleural or peritoneal catheter;
an elongated tubular portion extending from the connector with a lumen therein extending from a distal end of the tubular portion at the connector to a proximal end of the tubular portion;
a fluid container configured to be in fluid communication with the elongated tubular portion and to contain collected fluid therein; and
a pumping portion for pumping fluid from the pleural or peritoneal catheter to the fluid container, wherein the pumping portion, elongated tubular portion, and fluid container are permanently connected and intended to be disposed of after a drainage bag is filled or a drainage operation is completed.

2. The system of claim 1, wherein the pumping portion is an in-line pump configured to pump fluid through the elongated tubular portion.

3. The system of claim 1, wherein the pumping portion has a pump inlet with a first portion of the elongated tubular portion connected thereto and a pump outlet with a second portion of the elongated tubular portion connected thereto, wherein the system further comprising self-contained pump within a pump housing that powered by a battery contained within the pump housing.

4. The system of claim 1, wherein the pumping portion has an installable battery portion.

5. The system of claim 1, wherein the pump body includes a driving mechanism that mechanically drives the pumping mechanism within the pumping portion to pump fluid through the elongated tubular portion.

6. The system of claim 1, wherein the pumping portion comprises a section of the elongated tubular portion that is configured to be pinched to pump fluid through the elongated tubular portion.

7. The system of claim 1, wherein a rate of fluid flow though the elongated tubular portion is controllable via a pump controller that drives the pumping portion.

8. The system of claim 7, wherein the rate of fluid flow is controllable via a slider, dial, or switch.

9. The system of claim 1, further comprising a pressure sensor in one of the pumping portion, the elongated tubular portion, or the pumping portion, wherein an output of the pressure sensor is monitored and the pumping portion is controlled based on the output of the pressure sensor.

10. The system of claim 1, wherein the system further comprises one of a pH sensor, oxygen sensor, or glucose sensor for monitoring properties of the fluid pumped from the pleural or peritoneal catheter.

11. The system of claim 1, wherein the connector is configured to engage with a valve of the pleural or peritoneal catheter to allow fluid to flow therethrough.

12. The system of claim 1, wherein the fluid container is a bag.

13. The system of claim 1, wherein the pumping portion is configured to have a flow rate from 3ml/min to 400 ml/min.

14-15. (canceled)
